(19) United States
(12) Patent Application Publication
(10) Pub. No.: US 2010/0145381 A1
(43) Pub. Date: Jun. 10, 2010

(54) MULTI-RINGED SEPARATOR FOR TUBULAR ORGANS

(75) Inventor: Hyun-Joon Moon, Gwangju (KR)

Correspondence Address: LOWE HAUPTMAN HAM & BERNER, LLP, 1700 DIAGONAL ROAD, SUITE 300, ALEXANDRIA, VA, 22314, US

(73) Assignee: Hyun-Joon Moon, Gwangju (KR)

(21) Appl. No.: 11/909,445

(22) PCT Filed: Feb. 21, 2006
(86) PCT No.: PCT/KR06/00572

§ 371 Date: Nov. 26, 2007

(30) Foreign Application Priority Data
Mar. 22, 2005 (KR) 10-2005-0023694

(51) Int. Cl. A61B 17/28

(52) U.S. Cl. 606/207; 606/209

(57) ABSTRACT

A multi-ringed tubular organ separator includes a first arm having a handle at a rear end and a second arm having a handle at a rear end and combined with the first arm for rotation about a pivot into an opened position and a closed position. The first arm and the second arm are provided at their front ends with main clamps cooperating to define a main clamping zone in which a tubular organ and surrounding tissues of a mammal can be enclosed. The first arm and the second arm are further provided with sub clamps formed adjacent to the main clamps and cooperating to define a sub clamping zone in which the tubular organ and the surrounding tissues can be enclosed in isolation from the main clamping zone, the main clamping zone remaining in communication with the sub clamping zone through a throat.

### FIELD OF THE INVENTION

The present invention is directed to a multi-ringed tubular organ separator and, more specifically, to a multi-ringed tubular organ separator capable of easily and safely separating peripheral tissues and membranes surroundingly attached to a tubular organ when the tubular organ of a mammal is subjected to a surgical operation.

### BACKGROUND ART

Examples of tubular organs constituting the body of a mammal include blood vessels, nerves, ureters, bile ducts and vasa. The tubular organs are encompassed by a number of soft tissues, such as muscles and multi-layered fasciae, and surrounding tissues, such as nerves and blood vessels, extending around the tubular organs. In order to surgically operate such tubular organs, the surrounding tissues have to be peeled off in the process of cutting open the skin to create an incision window, searching for a target tubular organ through the incision window and conducting the aimed operation. Careful stanching is required at the time of finding out the tubular organ and peeling off and separating the surrounding tissues that encompass the tubular organ in multiple layers. This is because the blood vessels as well as the tissues are damaged in the above-noted process. The process of searching for and separating the target tubular organ to be operated involves difficult and complicated tasks, including skin incision, tissue peeling-off, stanching of damaged blood vessels, skin suture and so forth. This means that the operation of the tubular organs is time-consuming and requires the use of operating instruments of varying kinds and shapes. Representative examples of the tubular organ operation are a vasectomy and a vasectomy reversal.

In case of a conventional incisional vasectomy, one example of the vasectomy, local anesthesia is conducted for the area of a scrotum through which a vas passes and then a skin opening is created by incising a scrotal skin in one row or two rows along the length of the vas through the use of a surgical scalpel or an electric scalpel. Subsequently, the distal end of a ringed Allis tissue forceps or clamp with a ring size far greater than the diameter of the vas is inserted through the skin opening to grasp and take out the vas together with the surrounding tissues. Thereafter, the vas is exposed by stripping the surrounding tissues with a curved mosquito hemostat or the like instrument, while incising the surrounding tissues by means of the surgical scalpel. Then, a series of operating tasks such as transsection, ligation, galvanocautery, fascial interposition, suture of the skin opening and sterilization are performed depending on the surgeon's intention.

According to the conventional incisional vasectomy, however, the surrounding tissues peeled off from the vas should be clamped with the use of, e.g., a towel clamp, to make sure that the surrounding tissues are prevented from returning back to the original position and thus concealing the vas again during the course of operation. Use of the towel clamp or the like gives rise to a possibility that additional damage is caused to the surrounding tissues and the blood vessels. Furthermore, in view of the fact that the Allis tissue forceps has a relatively large size, it is necessary to correspondingly increase the size of the skin opening through which the Allis tissue forceps is inserted to grasp and take out the vas and the surrounding tissues. This makes it quite difficult to minimize the damage or incision of the skin and the surrounding tissues. Due to the increased size of the skin opening, a need exists to make suture of the opened skin and the stitching fibers used in the suture have to be removed at a later time, which tasks are cumbersome and inconvenient to do.

Another example of the vasectomy is a so-called no-scalpel vasectomy originating in the People's Republic of China and then propagated to Republic of Korea, U.S.A. and other countries. According to the no-scalpel vasectomy, the vas is searched for and placed in the vicinity of a scrotum midline, after which a local anesthetic is injected into the skin, the underlying tissues and the vas in a sufficient quantity to anesthetize them. Then, the vas is grasped and pulled up together with the skin, the hypodermic tissues and the surrounding tissues through the use of a ringed clamp specially designed for the no-scalpel vasectomy. Conducted next is a puncturing step wherein the skin is punctured with one of the sharp-edged end portions of a dissecting forceps. Subsequently, stripping is performed whereby the skin and the hypodermic tissues are stripped from the vas using both end portions of the dissecting forceps. Thereafter, the multi-layered surrounding tissues that encompass the vas are stripped one by one to separate the vas, at which time some surgeons often incise the skin, the hypodermic tissues and the surrounding tissues using a surgical scalpel or an electric scalpel.

Once the vas is separated from the surrounding tissues, it is taken out to the outside of the skin with the use of one end portion of the dissecting forceps, while relieving the clamping force of the ringed clamp. At this time, in order to more thoroughly peel off the surrounding tissues of the vas thus taken out, the vas is grasped once again with the ringed clamp and then the surrounding tissues around the vas are carefully and elaborately stripped with the blood vessels running adjacent to the vas left intact. Subsequently, a series of operating tasks such as transsection, ligation, galvanocautery, fascial interposition, suture of the skin opening and sterilization are carried out at the surgeon's will.

According to the no-scalpel vasectomy referred to above, however, it is highly likely that the vas is missed due to incorrect grasping because the vas has to be grasped by the ringed clamp together with the skin, the hypodermic tissues and the surrounding tissues. The vas may sometimes be partially grasped and lacerated. Particularly, there may be an instance that the stripped vas is slid down from the ringed clamp or severed by the sharp edge of the dissecting forceps, when the vas is taken out to the outside of the skin with the use of one end portion of the dissecting forceps, while relieving the clamping force of the ringed clamp. In this event, the vas is dropped into the scrotum and cannot be searched for with ease. Furthermore, it is likely that the dissecting forceps is inserted too deep and causes damage to the vas by piercing. Slight deviation of the direction in which the dissecting forceps is inserted may injure the blood vessels and the surrounding tissues. Unlike the typical patients, in case of a patient who has vasa inheritedly placed deep in a scrotum, a patient who has a thick scrotum skin or a small-sized or contracted scrotum and a patient who suffers from abdominal obesity, it is quite difficult for the ringed clamp to collectively clamp the vas together with the skin, the hypodermic tissues and the surrounding tissues. Instead, the blood vessels or the fasciae may be mistakenly recognized as the vas, which requires the surgeons to go through many trials and errors in the vas clamping process. This means that the no-scalpel vasectomy cannot be universally applied to each and every patient. Thus, in an effort to avoid erroneous operations and to minimize damage of the blood vessels and the surrounding tissues in an operation process, an ongoing demand has existed to provide the surgeons exercising the no-scalpel vasectomy with training or education or to give the surgeons an opportunity of undergoing many operation experiences.

### DETAILED DESCRIPTION OF THE INVENTION

### Technical Problems

In view of the above-noted problems inherent in the prior art, it is an object of the present invention to provide a multi-ringed tubular organ separator capable of collectively grasping and taking out a tubular organ and its surrounding tissues through a minimum-sized skin opening in an easy and safe manner, with minimized damage to blood vessels, nerves and tissues around the tubular organ.

Another object of the present invention is to provide a multi-ringed tubular organ separator that can easily separate a tubular organ from multi-layered surrounding tissues.

A further object of the present invention is to provide a multi-ringed tubular organ separator that can easily and correctly isolate a tubular organ and varying kinds of surrounding tissues respectively in a main clamping zone and a sub clamping zone which are disposed in succession.

### Solution to the Technical Problems

In order to achieve these objects, the present invention provides a multi-ringed tubular organ separator comprising: a first arm having a handle at a rear end; and a second arm having a handle at a rear end and combined with the first arm for rotation about a pivot into an opened position and a closed position, wherein the first arm and the second arm are provided at their front ends with main clamps cooperating to define a main clamping zone in which a tubular organ and surrounding tissues of a mammal can be enclosed, the main clamps having round contact portions at their distal ends, the round contact portions adapted to make contact with each other when the first arm and the second arm are closed, wherein the first arm and the second arm are further provided with sub clamps formed adjacent to the main clamps and cooperating to define a sub clamping zone in which the tubular organ and the surrounding tissues can be enclosed in isolation from the main clamping zone, the main clamping zone remaining in communication with the sub clamping zone through a throat.

### BRIEF DESCRIPTION OF THE DRAWINGS

FIG. 1 is a front elevational showing the overall configuration of a multi-ringed tubular organ separator in accordance with one embodiment of the present invention;

FIG. 2 is an enlarged front view illustrating clamps employed in the multi-ringed tubular organ separator of the present invention;

FIG. 3 is a cross-sectional view taken along line III-III in FIG. 2;

FIG. 4 is a cross-sectional view taken along line IV-IV in FIG. 2;

FIG. 5 is a perspective view depicting a condition where the multi-ringed tubular organ separator of the present invention is inserted through a skin opening to take out a vas and its surrounding tissues to the outside of a skin;

FIG. 6 is a perspective view illustrating a condition where the vas and the surrounding tissues are taken out to the outside of the skin by means of the multi-ringed tubular organ separator of the present invention;

FIG. 7 is a perspective view showing a condition where the vas and the surrounding tissues taken out by the multi-ringed tubular organ separator are raised up in order to strip the surrounding tissues from the vas;

FIG. 8 is a perspective view depicting a condition where the vas and the surrounding tissues are separated from each other and placed respectively in a main clamping zone and a sub clamping zone of the multi-ringed tubular organ separator of the present invention; and

FIG. 9 is a front elevational showing a multi-ringed tubular organ separator in accordance with another embodiment of the present invention.

### BEST MODE FOR CARRYING OUT THE INVENTION

Hereinafter, preferred embodiments of the present invention will be described in detail with reference to the accompanying drawings.

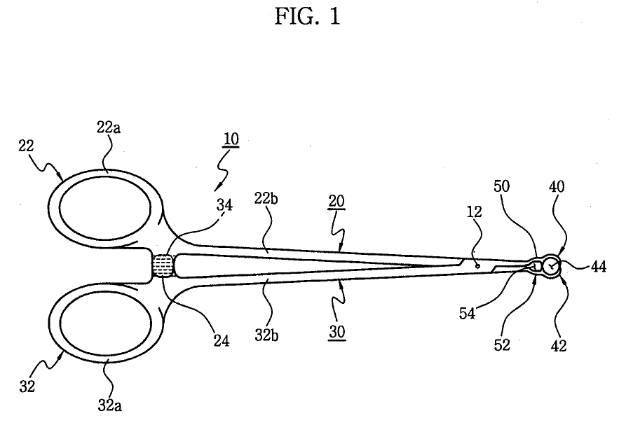

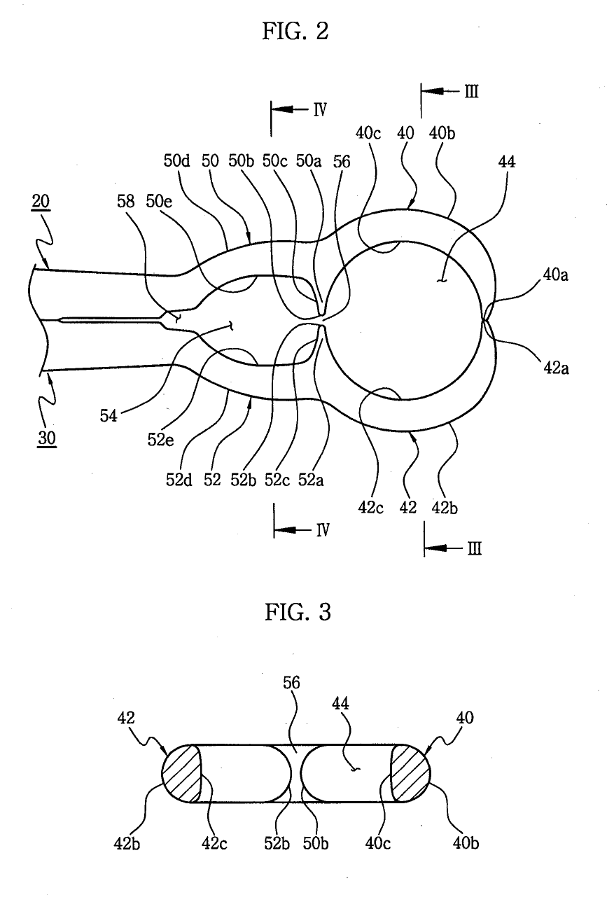

Referring first to FIGS. 1 and 2, a multi-ringed tubular organ separator 10 in accordance with the present invention includes a pair of first and second elongated arms 20 and 30. The first arm 20 and the second arm 30 are combined with each other for relative rotation about a pivot 12. The front ends of the first arm 20 and the second arm 30 are opened and closed in response to the scissor movement of the arms 20 and 30. The multi-ringed tubular organ separator shown in FIG. 1 is of a front leverage type in which the pivot 12 lies at a halfway point between the middle and the front ends of the first arm 20 and the second arm 30. The position of the pivot 12 may be properly changed if needed.

Provided at the rear end parts of the first arm 20 and the second arm 30 are handles 22 and 32 that can be gripped by one hand of a surgeon. The handles 22 and 32 are comprised of loop-shaped grip portions 22a and 32a into which the thumb and the fingers can be inserted to grip the same and lever-like grip portions 22b and 32b extending forward from the loop-shaped grip portions 22a and 32a. Mutually engageable toothed locks 24 and 34 are formed on the lever-like grip portions 22b and 32b in the vicinity of the loop-shaped grip portions 22a and 32a.

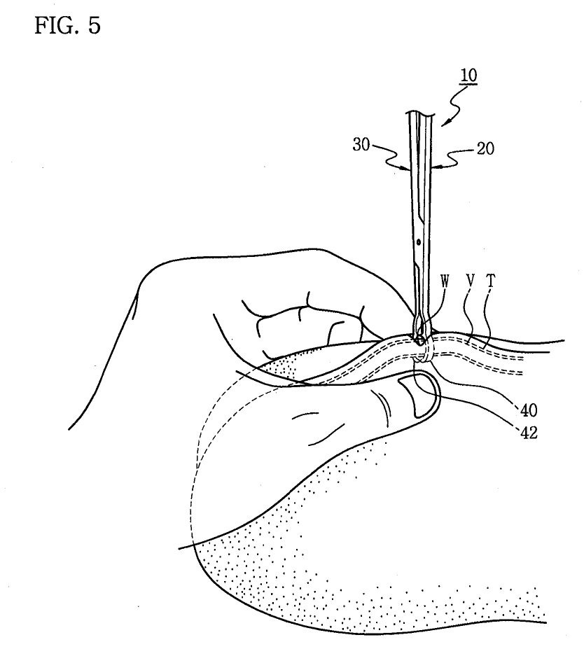

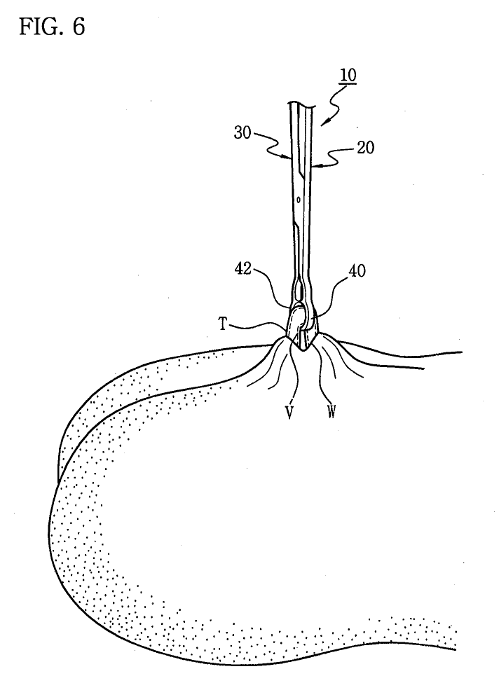

Referring to FIGS. 1 through 3, arcuate main clamps 40 and 42 are provided at the front ends of the first arm 20 and the second arm 30. Each of the main clamps 40 and 42 has a beak-shaped end extension whose sectional area is gradually reduced. The main clamps 40 and 42 are provided at their distal ends with round contact portions contact and 42a. If the locks 24 and 34 are engaged with each other and the first arm 20 and the second arm 30 are closed, the round contact portions 40a and 42a of the main clamps 40 and 42 make contact with each other to thereby form a main clamping zone 44. The round contact portions 40a and 42a are so dull that they cannot pierce, incise or injure a vas V, surrounding tissues T, blood vessels or the like at the time when the main clamps 40 and 42 are inserted through a skin opening W to grasp a tubular organ, e.g., the vas V, and the surrounding tissues T as illustrated in FIGS. 5 and 6.

The main clamps 40 and 42 have outer surfaces 40b and 42b of a convex longitudinal shape and inner surfaces 40c and 42c of a concave longitudinal shape. As can be seen in FIG. 3, the outer surfaces 40b and 42b has a semicircular cross-sectional shape with a relatively great curvature, while the inner surfaces 40c and 42c has a curved cross-sectional shape with a curvature smaller than that of the outer surfaces 40b and 42b. The shape and the curvature of the outer surfaces 40b and 42b and the inner surfaces 40c and 42c shown in FIG. 3 are for the illustrative purpose only and may be appropriately changed if such a need arises. Namely, the outer surfaces 40b and 42b may have an elliptical cross-sectional shape and the inner surfaces 40c and 42c may be of a nearly flat arcuate cross-sectional shape with an even smaller curvature. Forming the outer surfaces 40b and 42b in the semicircular cross-sectional shape as noted above ensures that the main clamps 40 and 42 make contact at a reduced area with the tissues and the blood vessels around the skin opening W second arm 30 so as not to cause damage to the tissues and the blood vessels at the time when the main clamps 40 and 42 are inserted through the skin opening W to grasp the vas V and the surrounding tissues T as illustrated in FIG. 5. The inner surfaces 40c and 42c of a curved cross-sectional shape make contact at an increased area with the vas V and the surrounding tissues T when the vas V and the surrounding tissues T are grasped and pulled up by means of the main clamps 40 and 42 and then separated from each other as illustrated in FIGS. 6 through 8. This helps to disperse the grasping force of the main clamps 40 and 42 applied to the vas V and the surrounding tissues T, thus reducing or precluding the possibility of injuring the vas V and the surrounding tissues T.

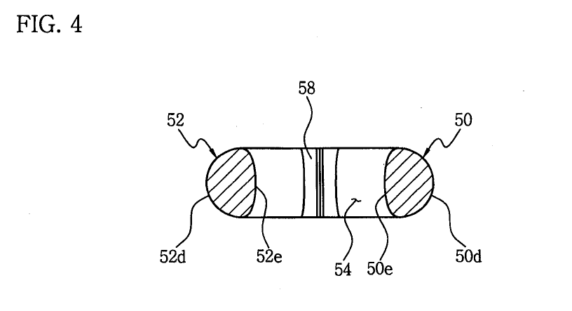

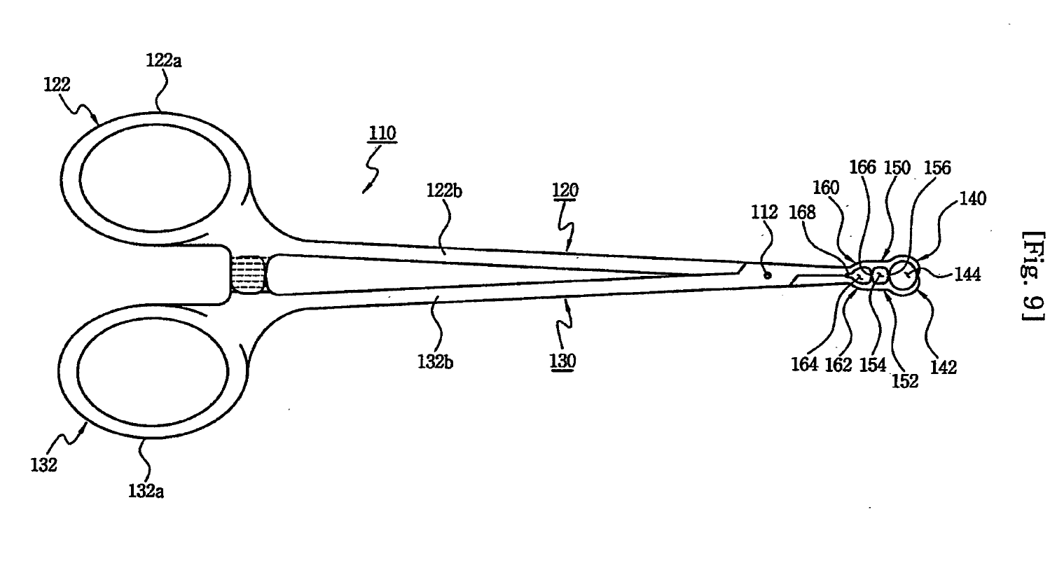

Referring to FIGS. 1, 2 and 4, arcuate sub clamps 50 and 52 are provided in succession respectively at the rear parts of the main clamps 40 and 42. The sub clamps 50 and 52 are adapted to create a sub clamping zone 54 when the first arm 20 and the second arm 30 are closed. The main clamping zone 44 has an area greater than that of the sub clamping zone 54 so that the main clamping zone 44 can receive a greater number of tubular organs and tissues than the sub clamping zone 54 does. As illustrated in FIGS. 5 through 7, the main clamping zone 44 can collectively enclose the vas V and the surrounding tissues T therein. As depicted in FIG. 8, the sub clamping zone 54 can isolate the surrounding tissues T stripped from the vas T. The main clamping zone 44 is shown in FIG. 2 to have a generally circular shape, with the sub clamping zone 54 having a generally elliptical shape. However, the shape of the main clamping zone 44 and the sub clamping zone 54 shown in FIG. 2 is for the illustrative purpose only, which means that the shape and area of the main clamping zone 44 and the sub clamping zone 54 may be arbitrarily changed if desired.

Referring again to FIGS. 1, 2 and 4, the main clamping zone 44 and the sub clamping zone 54 are divided by ridges 50a and 52a formed at the boundary where the main clamps 40 and 42 meet with the sub clamps 50 and 52. Formed between the ridges 50a and 52a is a throat 56 that allows the main clamping zone 44 to communicate with the sub clamping zone 54. Preferably, the throat 56 has a width smaller than the diameter of the vas V in order to make sure that only the surrounding tissues T stripped from the vas V can pass the throat 56. The ridges 50a and 52a are provided at their distal ends with sharpened tips 50b and 52b whose sharpness is so selected as to allow the surrounding tissues T to be naturally stripped from the vas V with no damage as they are moved into the sub clamping zone 54.

As shown in FIG. 3, the tips 50b and 52b of the ridges 50a and 52a have a convex shape when viewed from the main clamping zone 44. Each of the tips 50b and 52b tapers away toward its extremity and terminates at a front-most end of a round shape. Accordingly, in the process of moving the surrounding tissues T through the throat 56 defined between the ridges 50a and 52a, the surrounding tissues T are smoothly stripped and isolated from hydraulic excavator vas V without causing damage to the tissues and the blood vessels. As can be seen in FIG. 2, flat inclined surfaces 50c and 52c are formed at the sides of the ridges 50a and 52a facing the sub clamping zone 54. The flat inclined surfaces 50c and 52c helps to restrain any unwanted escapement of the surrounding tissues T from the sub clamping zone 54 to the main clamping zone 44.

Furthermore, the sub clamps 50 and 52 has outer surfaces 50d and 52d and inner surfaces 50e and 52e formed in an arcuate shape just like the outer surfaces 40b and 42b and the inner surfaces 40c and 42c. A recess 58 is formed at the rear part of the sub clamping zone 54. Under the state that the first arm 20 and the second arm 30 are closed, the recess 58 plays a part of a spare space that accommodates a part of the surrounding tissues T enclosed within the sub clamping zone 54. Another function of the recess 58 is to preclude the possibility that the surrounding tissues T are caught in the gap between the first arm 20 and the second arm 30.

Turning to FIG. 9, there is shown a multi-ringed tubular organ separator in accordance with another embodiment of the present invention. The basic configuration and operation of the multi-ringed tubular organ separator of this embodiment is substantially the same as that of the separator 10 set forth above in respect of the preceding embodiment. For this reason, no detailed description will be given to the same elements or components.

As illustrated in FIG. 9, the multi-ringed tubular organ separator 110 in accordance with another embodiment of the present invention includes a first arm 120 and a second arm 130, both of which can be rotated about a pivot 112 into an opened position and a closed position. Provided at the rear ends of the first arm 120 and the second arm 130 are handles 122 and 132 comprised of loop-shaped grip portions 122a and 132a and lever-like grip portions 122b and 132b.

The first arm 120 and the second arm 130 further have main clamps 140 and 142, first sub clamps 150 and 152, and second sub clamps 160 and 162 that are formed in succession from the front ends toward the rear ends of the first arm 120 and the second arm 130. If the first arm 120 and the second arm 130 are closed, a main clamping zone 144, a first sub clamping zone 154 and a second sub clamping zone 164 are created by virtue of the main clamps 140 and 142, the first sub clamps 150 and 152, and the second sub clamps 160 and 162, respectively. The main clamping zone 144 is in communication with the first sub clamping zone 154 through a first throat 156, while the first sub clamping zone 154 communicates with the second sub clamping zone 164 via a second throat 166. A recess 168 is formed at the rear part of the second sub clamping zone 164. The main clamping zone 144 has an area greater than that of the first sub clamping zone 154 so that the main clamping zone 144 can receive a greater number of tubular organs and tissues than the first sub clamping zone 154 does. The area of the first sub clamping zone 154 is substantially the same as that of the second sub clamping zone 164, although the former may be changed to become greater than the latter. Unlike the embodiment illustrated in FIG. 9 wherein two sub clamping zones are employed, the number of sub clamping zones may be increased if needed.

Now, description will be given to the function exercised by the multi-ringed tubular organ separator of the present invention set forth above. In this regard, vasectomy will be described as an example of surgical operation for a tubular organ.

Referring to FIGS. 1 and 5, the surgeon first lays a patient down and makes a series of preoperative preparations such as examination, sterilization and anesthesia of a scrotum. Then, a skin opening W is created by piercingly incising a scrotum skin 0.5 cm long and 1-1.5 cm deep through the use of an Iris scissors, a surgical scalpel or the like.

Subsequently, the surgeon grips and pulls up the skin portion around the skin opening W with one hand and then inserts about 5 cm depth a small straight mosquito hemostat in an upright posture through the skin opening W, after which the mosquito hemostat is removed to thereby form a passageway leading to the vas V and the surrounding tissues T underneath the scrotum skin.

The surgeon holds the handles 22 of the multi-ringed tubular organ separator 10 with the other hand and pushes the separator 10 vertically into the previously formed passageway through the skin opening W, at which time the main clamps 40 and 42 are kept closed. In this process, the surgeon grips the vas V against any movement with one hand and brings the main clamps 40 and 42 into the vicinity of the vas V as close as possible. The main clamps 40 and 42 are then opened slightly and the vas V is pushed into the main clamping zone 44, after which the main clamps 40 and 42 are closed. Thereafter, as illustrated in FIG. 6, the surgeon pulls out the multi-ringed tubular organ separator 10 so that the vas V and the surrounding tissues T grasped by the main clamps 40 and 42 can be taken out to the outside. Then, the presence of the vas V is visually confirmed by the surgeon.

By bringing the main clamps 40 and 42 into the vicinity of the vas V as close as possible to grasp the vas V and taking out the vas V to the outside of the scrotum skin in this manner, it becomes possible to minimize unnecessary damage which would otherwise caused to a dartos fascia, an external spermatic fascia, an internal spermatic fascia, a cremaster fascia and the soft tissues thereof lying between the scrotum skin and the vas V. In addition, it is possible to readily take out and visually confirm the vas V even in a case that the patient has a vas V placed deep in the scrotum or a relatively thin vas V. In particular, the visual confirmation of the vas V helps to preclude occurrence of such a medical accident that blood vessels or nerves having a diameter similar to that of the vas V are recognized to be the vas V and incised mistakenly.

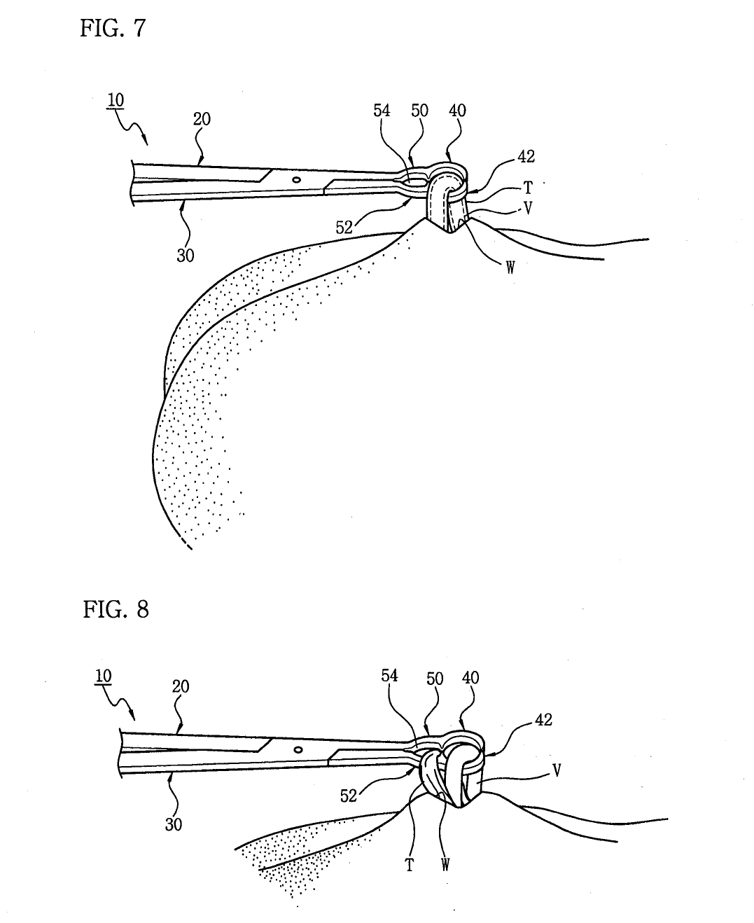

Next, as shown in FIG. 7, the surgeon lay down the multi-ringed tubular organ separator 10 so as to extend horizontally in a generally parallel relationship with the body of the patient, thus further pulling up the vas V grasped by the main clamps 40 and 42. Thereafter, as illustrated in FIG. 8, the surgeon strips the surrounding tissues T from the vas V little by little with a small curved hemostat. The surrounding tissues T thus stripped are moved from the main clamping zone 44 to the sub clamping zone 54 through the throat 56. In the process of moving the surrounding tissues T through the throat 56, the surrounding tissues T are naturally stripped from the vas V by means of the sharpened tips 50b and 52b of the ridges 50a and 52a. This makes it possible to strip the surrounding tissues T with no or little use of a surgical scalpel or an electric scalpel. Such an operation technique helps to minimize damage to the surrounding tissues T, as compared to the conventional operation wherein the surgical scalpel or the electric scalpel is used to incise and strip the surrounding tissues T from the vas V for separation of the vas V. As a result, it is possible to shorten the time taken in operation and restoration of the patient from the operative injury, and also to minimize occurrence of a sequela and a complication of operation.

Once the surrounding tissues T are completely moved into the sub clamping zone 54 of the sub clamps 50 and 52 thus leaving the vas V alone in the main clamping zone 44 of the main clamps 40 and 42, a series of operating tasks for the vas V such as transsection, ligation, galvanocautery, fascial interposition, suture of the skin opening and sterilization are performed depending on the surgeon's intention, thereby terminating the intended vasectomy.

In case of using the multi-ringed tubular organ separator 110 shown in FIG. 9, the tubular organ and the surrounding tissues are first enclosed within the main clamping zone 144 of the main clamps 140 and 142, and then the surrounding tissues T stripped from the tubular organ, the blood vessels, the nerves and the like are isolated in the first sub clamping zone 154 of the first sub clamps 150 and 152 and the second sub clamping zone 164 of the second sub clamps 160 and 162.

Although preferred embodiments of the present invention have been described in detail with reference to the accompanying drawings, it will be apparent to those skilled in the art that various changes or modifications may be made thereto within the scope of the invention defined by the appended claims. For example, the multi-ringed tubular organ separator in accordance with the present invention may be equally employed in performing a surgical operation for other tubular organs than a vas, e.g. a nerve system.

### INDUSTRIAL APPLICABILITY

As described in the foregoing, according to the multi-ringed tubular organ separator of the present invention, it is possible to collectively grasp and take out a tubular organ and its surrounding tissues through a minimum-sized skin opening in an easy and safe manner, with minimized damage to the tubular organ as well as soft tissues, blood vessels, nerves and surrounding tissues between the skin and the tubular organ. Furthermore, thank to the fact that the surrounding tissues are stripped and isolated merely by moving the surrounding tissues from a main clamping zone to a sub clamping zone, the tubular organ and varying kinds of surrounding tissues can be easily and correctly isolated respectively in the main clamping zone and the sub clamping zone. By separating the surrounding tissues from the tubular organ without resort to a surgical scalpel or an additional operative process and with minimized damage to the surrounding tissues, it is possible to shorten the time taken in operation and restoration of a patient from operative injury, and also to minimize occurrence of a sequela and a complication of operation.

1. A multi-ringed tubular organ separator comprising:
a first arm having a handle at a rear end; and
a second arm having a handle at a rear end and combined with the first arm for rotation about a pivot into an opened position and a closed position,
wherein the first arm and the second arm are provided at their front ends with main clamps cooperating to define a main clamping zone in which a tubular organ and surrounding tissues of a mammal can be enclosed, the main clamps having round contact portions at their distal ends, the round contact portions adapted to make contact with each other when the first arm and the second arm are closed,
wherein the first arm and the second arm are further provided with sub clamps formed adjacent to the main clamps and cooperating to define a sub clamping zone in which the tubular organ and the surrounding tissues can be enclosed in isolation from the main clamping zone, the main clamping zone remaining in communication with the sub clamping zone through a throat.

2. The multi-ringed tubular organ separator as recited in claim 1, wherein the main clamping zone and the sub clamping zone are divided by ridges formed at a boundary between the main clamps and the sub clamps, the ridges spaced apart from each other to define the throat between the ridges, the ridges having sharpened tips for helping the surrounding tissues to be stripped from the tubular organ.

3. The multi-ringed tubular organ separator as recited in claim 1, wherein the throat has a width smaller than a diameter of the tubular organ so as to allow the surrounding tissues alone to be moved through the throat, the ridges having flat inclined surfaces for restraining escapement of the surrounding tissues from the sub clamping zone to the main clamping zone.

4. The multi-ringed tubular organ separator as recited in claim 3, wherein the main clamping zone is of a circular shape and the sub clamping zone is of a generally elliptical shape.

5. The multi-ringed tubular organ separator as recited in claim 3, wherein the main clamping zone has an area greater than that of the sub clamping zone.

6. The multi-ringed tubular organ separator as recited in claim 3, wherein the sub clamping zone has a recess at a rear part, which serves as a spare space.

7. The multi-ringed tubular organ separator as recited in claim 3, wherein each of the tips of the ridges has a convex shape when viewed from the main clamping zone.

8. The multi-ringed tubular organ separator as recited in claim 1, wherein each of the main clamps and the sub clamps has a convex inner cross-sectional surface and a convex outer cross-sectional surface, the inner cross-sectional surface having a curvature smaller than that of the outer cross-sectional surface.

9. The multi-ringed tubular organ separator as recited in claim 8, wherein the outer cross-sectional surface is of a semicircular shape.

10. The multi-ringed tubular organ separator as recited in claim 1, wherein the first arm and the second arm are further provided with additional clamps lying adjacent to the sub clamps to define an additional clamping zone.
